(19) United States
(12) Patent Application Publication
(10) Pub. No.: US 2026/0272326 A1
(43) Pub. Date: Sep. 17, 2026

(54) NASAL RESPIRATION MEASUREMENT DEVICE

(71) Applicants: SEIKO GROUP CORPORATION, Tokyo (JP); Science Energy CO., LTD., Tokyo (JP)

(72) Inventors: Kazumasa SASAHARA, Matsudo-shi (JP); Yoshifumi YOSHIDA, Matsudo-shi (JP); Kotaro MAKI, Tokyo (JP)

(21) Appl. No.: 19/168,709

(22) PCT Filed: Feb. 27, 2024
(86) PCT No.: PCT/JP2024/006948

§ 371 Date: Sep. 24, 2025

(30) Foreign Application Priority Data
Mar. 27, 2023 (JP) 2023-050007

(51) Int. Cl. A61B 5/087; A61B 5/097

(52) U.S. Cl. CPC A61B 5/0876; A61B 5/097; A61B 2562/0247

(57) ABSTRACT

A nasal respiration measurement device includes a base member in which a measurement flow path that is in fluid communication with a nostril of a user is formed to pass through the base member, and a pressure resistance portion that protrudes from an inner wall surface of the measurement flow path.

### RELATED APPLICATIONS

This application is a 371 application of PCT/JP2024/006948 having an international filing date of Feb. 27, 2024, which claims priority to Japanese Patent Application No. 2023-050007, filed Mar. 27, 2023, the entire content of each of which is incorporated herein by reference.

### TECHNICAL FIELD

The present invention relates to a nasal respiration measurement device.

### BACKGROUND ART

Patent Document 1 discloses a respiratory flow rate measurement device that measures a respiratory flow rate of a subject. The respiratory flow rate measurement device includes a nasal cavity contact body that is brought into contact with a nasal cavity of a subject and communicates a respiratory flow from the nasal cavity to the inside of the nasal cavity contact body.

### CITATION LIST

### Patent Document

Patent Document 1: Japanese Unexamined Patent Application, First Publication No. 2021-35441

### SUMMARY OF INVENTION

### Technical Problem

The nasal cavity contact body is formed in a substantially truncated cone shape with a hollow inside, and includes a pair of nasal cavity contact portions with outer peripheries sealing a pair of nasal cavities of the subject in an airtight manner. In a case where a pressure sensor measures exhaled air flowing inside the nasal cavity contact portion, there is a problem in that the pressure sensor does not respond as expected to an exhaled air flow as compared with an inhaled air flow.

The present invention is made in view of the above-described problems, and an object of the present invention is to provide a nasal respiration measurement device that can change an exhaled air flow in nasal respiration of a user and measure the respiratory flow with high accuracy.

### Solution to Problem

(1): A nasal respiration measurement device according to an aspect of the present invention includes: a base member in which a measurement flow path that is in fluid communication with a nostril of a user is formed to pass through the base member; and a pressure resistance portion that protrudes from an inner wall surface of the measurement flow path.

With the nasal respiration measurement device according to the present aspect, an exhaled air flow collides with the pressure resistance portion protruding from the inner wall surface of the measurement flow path, and the exhaled air flow is changed. Therefore, it is possible to measure the exhaled air flow with the pressure sensor.

Therefore, it is possible to measure the exhaled air flow, and the respiratory flow can be measured with high accuracy.
(2): In the nasal respiration measurement device according to Aspect (1), the pressure resistance portion may be formed integrally with the base member.

In this case, it is possible to reduce the cost by reducing the number of components.
(3): In the nasal respiration measurement device according to Aspect (1) or (2), a plurality of the pressure resistance portions may be provided on the inner wall surface of the measurement flow path.

In this case, since the exhaled air flow can be changed at a plurality of places, measurement sensitivity of the exhaled air flow is improved.
(4): In the nasal respiration measurement device according to any one of Aspects (1) to (3), a plurality of the pressure resistance portions may be provided on the inner wall surface of the measurement flow path in series with respect to a fluid flow of the measurement flow path.

In this case, since the exhaled air flow can be changed at a plurality of places along an exhaled air flow, measurement of the exhaled air flow is stabilized.
(5): In the nasal respiration measurement device according to any one of Aspects (1) to (4), the pressure resistance portion may be provided in an annular shape over an entire inner wall surface of the measurement flow path.

In this case, the exhaled air flow can be changed over the entire measurement flow path.
(6): In the nasal respiration measurement device according to any one of Aspects (1) to (5), the pressure resistance portion may have an asymmetric shape between an upstream side and a downstream side thereof.

In this case, it is possible to apply different rectification (change in flow) actions to the exhaled air flow and the inhaled air flow.
(7): In the nasal respiration measurement device according to any one of Aspects (1) to (6), a communication port communicating with a space in which a pressure sensor is disposed may be formed in the inner wall surface of the measurement flow path.

In this case, a position where the pressure is measured can be easily changed depending on the position of the communication port formed in the inner wall surface of the measurement flow path.
(8): In the nasal respiration measurement device according to Aspect (7), the pressure sensor may include a substrate portion disposed perpendicular to an axial direction in which a central axis of the measurement flow path extends.

In this case, since the axial direction of the measurement flow path and the thickness direction of the substrate portion match each other, a dimension of the nasal respiration measurement device in the axial direction can be reduced.
(9): In the nasal respiration measurement device according to Aspect (7), the pressure sensor may include a substrate portion disposed in parallel with an axial direction in which a central axis of the measurement flow path extends.

In this case, since the axial direction of the measurement flow path and a plane direction of the substrate portion match each other, a radial dimension of the nasal respiration measurement device can be reduced.
(10): In the nasal respiration measurement device according to any one of Aspects (7) to (9), the measurement flow path may include, as the communication port, one or a plurality of first communication ports that are disposed in an upstream region on an upstream side of the pressure resistance portion during exhalation and communicate with a space in which a first measurement unit of the pressure sensor is disposed.

In this case, it is possible to determine not only a respiratory flow rate but also the exhaled air flow.
(11): In the nasal respiration measurement device according to Aspect (10), the measurement flow path may include, as the communication port, one or a plurality of second communication ports that are disposed in a downstream region on a downstream side of the pressure resistance portion during exhalation and communicate with a space in which a second measurement unit of the pressure sensor is disposed.

In this case, the accuracy of measurement is increased by taking a difference between outputs of the first measurement unit and the second measurement unit.
(12): In the nasal respiration measurement device according to Aspect (11), a pressure loss of the first communication port and a pressure loss of the second communication port may be different from each other.

Since the first communication port has a positive pressure during exhalation, in a case where the pressure loss of the first communication port is lower than the pressure loss of the second communication port, the exhaled air is more likely to enter the first communication port, and the sensitivity during exhalation is increased.

In addition, in a case where the pressure resistance of the pressure resistance portion is unintentionally increased due to an influence of nasal mucus or the like, a pressure applied to the first measurement unit during exhalation is increased, and a large pressure is unintentionally applied to the pressure sensor. Therefore, in a case where the pressure loss of the first communication port is higher than the pressure loss of the second communication port, the exhaled air is less likely to enter the first communication port, and damage to the pressure sensor can be prevented.
(13): In the nasal respiration measurement device according to any one of Aspects (7) to (12), the pressure resistance portion may be partially provided on the inner wall surface of the measurement flow path on a side of the communication port.

In this case, since the pressure resistance portion is disposed only in the minimum necessary region on the inner wall surface of the measurement flow path on the side of the communication port, the user can easily breathe.
(14): The nasal respiration measurement device according to any one of Aspects (7) to (13) may further include an exhaled air flow guide portion that is provided on the inner wall surface of the measurement flow path on a side opposite to the communication port and on an upstream side of the communication port during exhalation, and configured to guide exhaled air flowing on the side opposite to the communication port to a side of the communication port.

In this case, since the exhaled air flow, which is biased to the side opposite to the communication port, can be guided to the side of the communication port on the inner wall surface of the measurement flow path, the measurement sensitivity of the exhaled air flow is improved.
(15): In the nasal respiration measurement device according to Aspect of (14), a slope that mitigates pressure resistance of the exhaled air flow guide portion during inhalation may be provided on the inner wall surface of the measurement flow path.

In this case, even in a case where the exhaled air flow guide portion is provided, since the pressure resistance during inhalation in the measurement flow path can be mitigated, the user can easily breathe.
(16): In the nasal respiration measurement device according to Aspect of (14) or (15), the exhaled air flow guide portion may be installed consecutively to the pressure resistance portion.

In this case, since the exhaled air flow guide portion and the pressure resistance portion can be integrally formed, it is easy to manufacture the base member, and the manufacturing cost can be reduced.
(17): In the nasal respiration measurement device according to any one of Aspects (14) to (16), the base member may include an insertion portion that is inserted into the nostril, the measurement flow path may be formed to pass through the insertion portion, and the exhaled air flow guide portion may be disposed inside the insertion portion.

In this case, since the exhaled air flow guide portion disposed inside the insertion portion guides the exhaled air flowing into the measurement flow path to the side of the communication port, the measurement sensitivity of the exhaled air flow is improved.
(18): In the nasal respiration measurement device according to any one of Aspects (1) to (17), the measurement flow path may include an upstream region on an upstream side of the pressure resistance portion during exhalation and a downstream region on a downstream side of the pressure resistance portion during exhalation, and a flow path length of the downstream region may be shorter than a flow path length of the upstream region.

In this case, since the downstream region hardly affects the measurement of the exhaled air flow, the nasal respiration measurement device can be reduced in size by making the flow path length of the downstream region shorter than the flow path length of the upstream region.
(19): In the nasal respiration measurement device according to any one of Aspects (1) to (18), the measurement flow path may include an upstream region on an upstream side of the pressure resistance portion during exhalation, and a spiral protrusion is formed on an inner wall surface of the upstream region.

In this case, since the biased exhaled air flow along the inner wall surface of the measurement flow path can be stirred by the spiral protrusion, the measurement sensitivity of the exhaled air flow is improved.
(20): In the nasal respiration measurement device according to any one of Aspects (1) to (19), one or a plurality of through-holes may be formed in the pressure resistance portion.

In this case, even in a case where the pressure resistance portion is provided, since the through-hole is formed in the pressure resistance portion, the user can easily breathe.
(21): A nasal respiration measurement device according to another aspect of the present invention includes: a base member in which a measurement flow path that is in fluid communication with a nostril of a user is formed to pass through the base member, and a pressure resistance portion that protrudes from an inner wall surface of the measurement flow path, in which a communication port communicating with a space in which a pressure sensor is disposed is formed in the inner wall surface of the measurement flow path, and the pressure resistance portion has an asymmetric shape between an upstream side and a downstream side thereof.

According to this configuration, since exhalation and inhalation in the nasal respiration of the user can be changed differently, the respiratory flow can be measured with high accuracy.
(22): A nasal respiration measurement device according to still another aspect of the present invention includes: a base member in which a measurement flow path that is in fluid communication with a nostril of a user is formed to pass through the base member; and a pressure resistance portion that protrudes from an inner wall surface of the measurement flow path, in which a communication port communicating with a space in which a pressure sensor is disposed is formed in the inner wall surface of the measurement flow path, the pressure resistance portion is disposed on the inner wall surface of the measurement flow path on a downstream side of the communication port during exhalation, and the pressure resistance portion has an asymmetric shape between an upstream side and a downstream side thereof.

According to this configuration, since exhalation and inhalation in the nasal respiration of the user can be changed differently, the respiratory flow can be measured with high accuracy.
(23): A nasal respiration measurement device according to still another aspect of the present invention includes: a base member in which a measurement flow path that is in fluid communication with a nostril of a user is formed to pass through the base member; a pressure resistance portion that protrudes from an inner wall surface of the measurement flow path, in which a communication port communicating with a space in which a pressure sensor is disposed is formed in the inner wall surface of the measurement flow path, the pressure resistance portion is disposed on the inner wall surface of the measurement flow path on a downstream side of the communication port during exhalation and is partially provided on the inner wall surface of the measurement flow path on a side of the communication port, and the pressure resistance portion has an asymmetric shape between an upstream side and a downstream side thereof.

According to this configuration, the exhaled air flow in the nasal respiration of the user is changed, and the respiratory flow can be measured with high accuracy.

Advantageous Effects of Invention According to the aspects of the present invention, it is possible to obtain a nasal respiration measurement device that can change an exhaled air flow in nasal respiration of a user and can measure a respiratory flow with high accuracy.

### BRIEF DESCRIPTION OF DRAWINGS

FIG. 1 is a front view representing a state in which a nasal respiration measurement device according to a first embodiment is mounted on a nose of a user.

FIG. 2 is a perspective view of a nasal respiration measurement device according to the first embodiment.

FIG. 3 is a cross-sectional view taken along line III-III shown in FIG. 2.

FIG. 4 is a view showing measurement results of a pressure sensor 2 in a case where a pressure resistance portion is present (Example) according to the first embodiment and in a case where the pressure resistance portion is absent (Comparative Example).

FIG. 5 is a cross-sectional configuration diagram of a nasal respiration measurement device according to a second embodiment.

FIG. 6 is a cross-sectional configuration diagram of a nasal respiration measurement device according to a third embodiment.

FIG. 7 is a cross-sectional configuration diagram of a nasal respiration measurement device according to a fourth embodiment.

FIG. 8 is a view showing measurement results of the nasal respiration measurement device according to the fourth embodiment.

FIG. 9 is a cross-sectional configuration diagram of a nasal respiration measurement device according to a fifth embodiment.

FIG. 10 is a cross-sectional configuration diagram of a nasal respiration measurement device according to a sixth embodiment.

FIG. 11 is a cross-sectional configuration diagram of a nasal respiration measurement device according to a seventh embodiment.

FIG. 12 is a cross-sectional configuration diagram of a nasal respiration measurement device according to an eighth embodiment.

FIG. 13 is a cross-sectional configuration diagram of a nasal respiration measurement device according to a ninth embodiment.

FIG. 14 is a perspective view of a nasal respiration measurement device according to a tenth embodiment.

FIG. 15 is a cross-sectional configuration diagram of the nasal respiration measurement device according to the tenth embodiment.

FIG. 16 is a cross-sectional configuration diagram of a nasal respiration measurement device according to an eleventh embodiment.

FIG. 17 is a cross-sectional configuration diagram of a nasal respiration measurement device according to a twelfth embodiment.

FIG. 18 is a cross-sectional configuration diagram of a nasal respiration measurement device according to a thirteenth embodiment.

FIG. 19 is a cross-sectional configuration diagram of a nasal respiration measurement device according to a modification example of the thirteenth embodiment.

FIG. 20 is a cross-sectional configuration diagram of a nasal respiration measurement device according to a fourteenth embodiment.

FIG. 21 is a cross-sectional configuration diagram of a nasal respiration measurement device according to a modification example of the fourteenth embodiment.

### DESCRIPTION OF EMBODIMENTS

Hereinafter, embodiments according to the present invention are described with reference to the drawings.

### First Embodiment

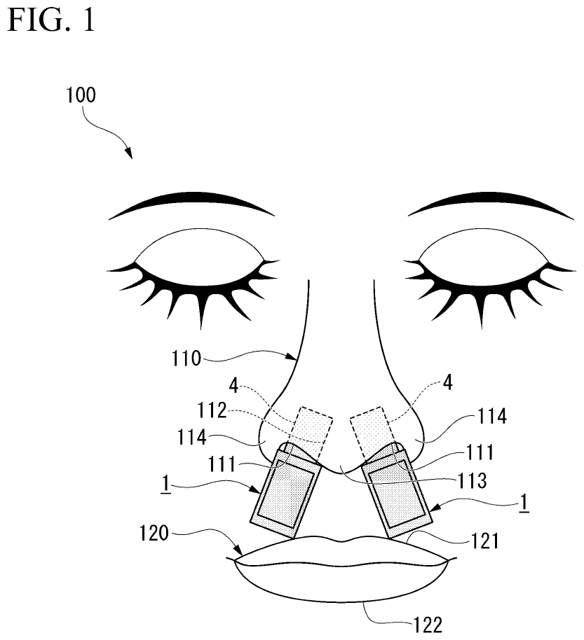

FIG. 1 is a front view showing a state in which a nasal respiration measurement device I according to a first embodiment is mounted on a nose 110 of a user 100.

As shown in FIG. 1, the nasal respiration measurement device 1 is mounted on right and left nostrils 111 of the nose 110 of the user 100.

It is noted that in FIG. 1, a reference numeral 100 indicates a user, a reference numeral 110 indicates a nose of the user, and a reference numeral 120 indicates a mouth of the user. The nose 110 includes the right and left nostrils 111, a nasal septum 112 that separates the right and left nostrils 111, a nasal tip 113 that is a distal end of the nose 110, and nasal wings 114 that bulge to the right and left of the nose 110. The mouth 120 includes an upper lip 121 and a lower lip 122.

In the nasal respiration measurement device 1, a portion exposed to the outside of each of the nostrils 111 in a state where the nasal respiration measurement device 1 is mounted on the nostrils 111 has a size that fits between the nose 110 and the upper lip 121 of the user 100. That is, the exposed portion has a size equal to or smaller than a length (for example, 20 mm) of the philtrum of the user 100. Preferably, the exposed portion is approximately 10 mm, and the total height (dimension in an upper and lower direction) of the device is approximately 20 mm. Accordingly, it is possible to suppress the nasal respiration measurement device 1 from blocking the mouth 120 of the user 100 or interfering with eating or speaking.

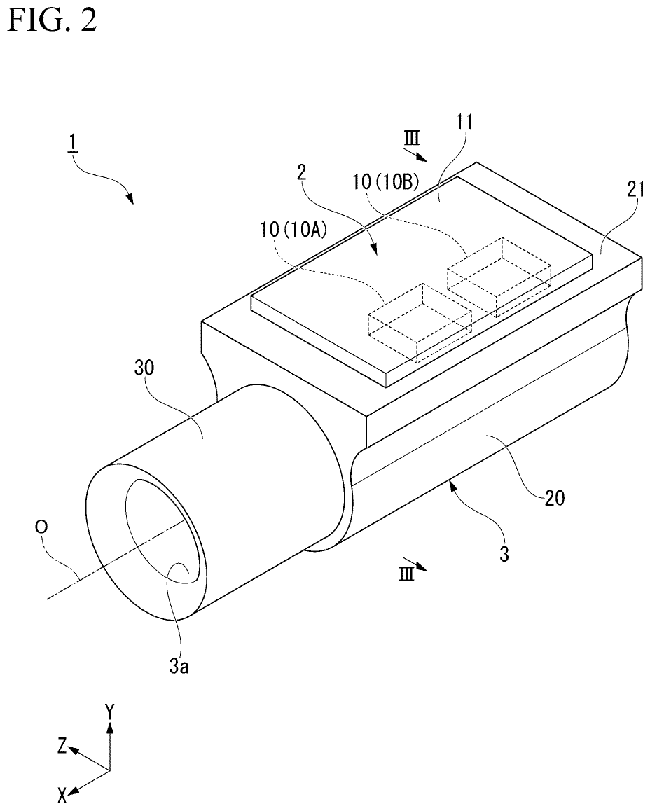

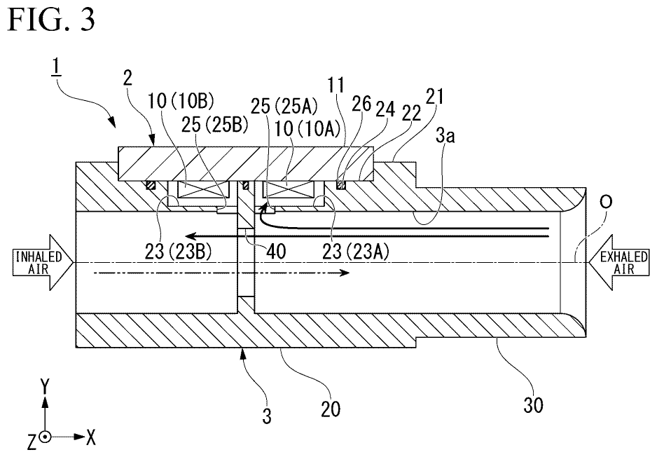

FIG. 2 is a perspective view of the nasal respiration measurement device 1 according to the first embodiment. FIG. 3 is a cross-sectional view taken along line III-III shown in FIG. 2.

As shown in the drawings, the nasal respiration measurement device 1 includes a pressure sensor 2 and a base member 3 to which the pressure sensor 2 is attached and in which a measurement flow path 3a that is in fluid communication with the nostril 111 is formed.

In the following description, an XYZ Cartesian coordinate system is set, and a configuration and a positional relationship of members may be described with reference to the XYZ Cartesian coordinate system. It is noted that an X-axis direction is an axial direction in which a central axis O of the measurement flow path 3a extends. A Y-axis direction is a radial direction orthogonal to an axial direction in which the central axis O of the measurement flow path 3a extends, and a-Y side is a side of the human body. A Z-axis direction is a radial direction orthogonal to an axial direction in which the central axis O of the measurement flow path 3a extends, and for example, a +Z side is a side of the nasal wing 114, and a −Z side is a side of the nasal septum 112.

Hereinafter, for convenience of description, a side of the pressure sensor 2 with respect to the base member 3 may be referred to as an upper side (+Y side), and a side opposite to the pressure sensor 2 with respect to the base member 3 may be referred to as a lower side (−Y side). It is noted that the +Y side may not be the upper side in a gravity direction.

As shown in FIG. 2, the pressure sensor 2 includes a measurement unit 10 and a substrate portion 11. The substrate portion 11 is, for example, a printed circuit substrate. A first measurement unit 10A and a second measurement unit 10B are provided as the measurement unit 10 on a lower surface side of the substrate portion 11 to be spaced apart from each other in the X-axis direction. It is noted that in addition to the measurement unit 10, a power supply unit (not shown) and a communication unit (not shown) are provided in the substrate portion 11.

The power supply unit supplies power to the measurement unit 10 and the communication unit. The power supply unit is, for example, an air cell. The air cell is a battery in which oxygen in the air is used as a positive electrode active material and a metal is used as a negative electrode active material, and since the active material on the positive electrode side is oxygen, it is not necessary to fill the positive electrode active material in a battery container, and it is suitable for a small size and long-term use. It is noted that the power supply unit may be a normal primary battery in which a positive electrode active material is filled in the battery container or a chargeable secondary battery.

The communication unit is a wireless communication module that performs wireless communication with an external device (not shown). The external device is, for example, a data processing device that receives a measurement result of the measurement unit 10 and calculates a state (flow rate and the like) of nasal respiration of the user 100. The nasal respiration measurement device 1 may include a storage unit that stores the measurement result of the measurement unit 10 instead of the communication unit or in combination with the communication unit. The storage unit may be, for example, a non-volatile memory, and the measurement result stored in the memory may be extracted in a case where the device is detached from the user 100.

The measurement unit 10 measures a pressure change due to a flow of air in the measurement flow path 3a in association with nasal respiration of the user 100. As a type of the measurement unit 10, for example, a resistance film type, a capacitance type, a piezoelectric element type, an optical type, a micro electro-mechanical system (MEMS) type, or the like can be employed. The measurement unit 10 may be used in combination with other sensors, for example, a humidity sensor, an acceleration sensor, a gyro sensor, a pulse wave sensor, or a flow rate sensor.

The base member 3 includes a base portion 20 to which the pressure sensor 2 is attached, and an insertion portion 30 that extends from the base portion 20 and is inserted into the nostril 111. An accommodation groove 22 that accommodates the substrate portion 11 is formed in an upper surface 21 of the base portion 20. The accommodation groove 22 is formed in a rectangular shape in plan view when viewed in the Y-axis direction similarly to the substrate portion 11.

As shown in FIG. 3, a chamber 23 that accommodates the measurement unit 10 is formed in the accommodation groove 22. As the chamber 23, a first chamber 23A that accommodates the first measurement unit 10A and a second chamber 23B that accommodates the second measurement unit 10B are formed in the accommodation groove 22 to be spaced apart from each other in the X-axis direction. The chamber 23 is formed in a rectangular shape in plan view when viewed from the Y-axis direction.

A groove portion 24 is formed in a peripheral edge portion of an opening of the chamber 23. A sealing member 26 is disposed in the groove portion 24. The sealing member 26 is in close contact with the lower surface of the substrate portion 11 and airtightly maintains the inside of the chamber 23. As the sealing member 26, a rubber member that is elastically deformable can be exemplified. It is noted that the sealing member 26 may be an adhesive, an adhesive gel, or the like.

The chamber 23 has a volume larger than that of the measurement unit 10. A communication port 25 communicating with a space (chamber 23) in which the measurement unit 10 is disposed is formed in an inner wall surface of the measurement flow path 3a. As the communication port 25, a first communication port 25A communicating with the first chamber 23A and a second communication port 25B communicating with the second chamber 23B are formed in the inner wall surface of the measurement flow path 3a to be spaced apart from each other in the X-axis direction.

The insertion portion 30 is formed in a cylindrical shape and extends from the base portion 20 to a +X side. The measurement flow path 3a is formed inside the insertion portion 30. The measurement flow path 3a passes through the insertion portion 30 and the base portion 20 in the X-axis direction. An inner wall surface of the measurement flow path 3a is formed in a cylindrical shape extending in the X-axis direction.

The base member 3 may have rigidity that maintains the shape of the measurement flow path 3a even in a case of being mounted on the nostril 111. The base member 3 is formed of, for example, a lightweight plastic material. It is noted that an elastic member (silicone rubber or the like) that protects a nostril mucous membrane may be attached to an outer peripheral surface of the insertion portion 30.

A pressure resistance portion 40 is provided on an inner wall surface of the measurement flow path 3a. The pressure resistance portion 40 is formed integrally with the base member 3 and protrudes inward in the radial direction from the inner wall surface of the measurement flow path 3a. The pressure resistance portion 40 of the present embodiment is provided in an annular shape over the entire inner wall surface of the measurement flow path 3a. The aperture diameter ratio of the pressure resistance portion 40 with respect to the measurement flow path 3a (an inner diameter of the pressure resistance portion 40/an inner diameter of the measurement flow path 3 a) may be 0.67 or more in order to obtain a rectifying effect.

The pressure resistance portion 40 is disposed on the inner wall surface of the measurement flow path 3a on a downstream side of the first communication port 25A and on an upstream side of the second communication port 25B during exhalation. That is, the pressure resistance portion 40 is disposed between the first communication port 25A and the second communication port 25B on the inner wall surface of the measurement flow path 3a. It is noted that in order to sufficiently exhibit an operational effect (described later) of the pressure resistance portion 40, it is preferable that a distance between the first communication port 25A and the pressure resistance portion 40 in the axial direction (X-axis direction) of the measurement flow path 3a is equal to or less than the diameter of the measurement flow path 3a.

During exhalation, in the measurement flow path 3a that is in fluid communication with the nostril 111, as indicated by a solid line arrow in FIG. 3, an exhaled air flow is generated from an opening end of the measurement flow path 3a on a side of the insertion portion 30 toward an opening end of the measurement flow path 3a on the side of the base portion 20 (toward the −X side).

On the other hand, during inhalation, as indicated by a two-dot chain line arrow in FIG. 3, an inhaled air flow of an intake flow (a flow toward +X side) is generated from the opening end of the measurement flow path 3a on the side of the base portion 20 to the opening end of the measurement flow path 3a on the side of the insertion portion 30.

A surface of the pressure resistance portion 40 facing the +X side is continuous with an inner wall surface on a downstream side during exhalation (an upstream side during inhalation) of the first communication port 25A in the Y-Z plane. In addition, a surface of the pressure resistance portion 40 facing the −X side is continuous with an inner wall surface on an upstream side during exhalation (a downstream side during inhalation) of the second communication port 25B in the Y-Z plane.

With the nasal respiration measurement device 1 having the above-described configuration, in a case where the user 100 performs nasal respiration, air flows through the measurement flow path 3a. As shown in FIG. 3, the communication port 25 communicating with the chamber 23 is formed in the inner wall surface of the measurement flow path 3a, and the measurement unit 10 can measure a pressure change of the measurement flow path 3a associated with nasal respiration at a position away from a mainstream of nasal respiration. Then, the flow rate of respiration can be measured by outputting a measurement result of the pressure change to an external device.

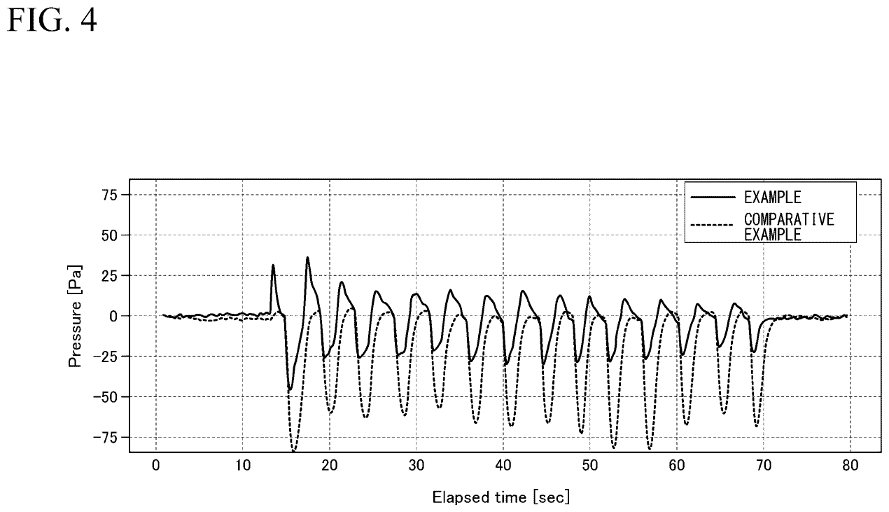

FIG. 4 is a view showing measurement results of the pressure sensor 2 in a case where the pressure resistance portion 40 according to the first embodiment is provided (example) and a case where the pressure resistance portion 40 is not provided (comparative example). Specifically, measurement is performed by the nasal respiration measurement device 1 with the pressure resistance portion 40 and the nasal respiration measurement device 1 without the pressure resistance portion 40 at the right and left nostrils 111. In FIG. 4, the vertical axis indicates a pressure, and the horizontal axis indicates time, In a case where the pressure is positive (+), it is exhalation. In a case where the pressure is negative (−), it is inhalation. It is noted that in FIG. 4, output results of the first measurement unit 10A of the pressure sensor 2 disposed in an upstream region on the upstream side of the pressure resistance portion 40 during exhalation are shown. In addition, in FIG. 4, a change in an absolute value is due to an influence of a nasal cycle.

As shown by a dotted line in FIG. 4, in the comparative example in which the pressure resistance portion 40 is not provided, the pressure is hardly output to the positive (+) side. That is, it can be seen that the pressure sensor 2 hardly reacts to the exhaled air flow in a case where the pressure resistance portion 40 is absent. On the other hand, as shown by the solid line in FIG. 4, in the example in which the pressure resistance portion 40 is provided, the pressure is output to the positive (+) side. That is, it can be seen that the pressure sensor 2 reacts to the exhaled air flow in a case where the pressure resistance portion 40 is present.

In this way, since the pressure resistance portion 40 is provided, it is possible to measure the exhaled air flow by the pressure sensor 2. Further, since the pressure resistance portion 40 is provided, the signs of a pressure measured with the exhaled air flow and a pressure measured with the inhaled air flow is different, it is possible to determine the exhaled air flow and the inhaled air flow. On the other hand, although the output on the negative (−) side is decreased with respect to the inhaled air flow, the pressure sensor 2 reacts to the inhaled air flow without any problem. That is, since the pressure resistance portion 40 is provided, the pressure sensor 2 does not stop responding to the inhaled air flow.

As shown in FIG. 3, a factor that enables measurement of the exhaled air flow by the pressure sensor 2 by providing the pressure resistance portion 40 is considered to be that the exhaled air flow collides with the pressure resistance portion 40 protruding from the inner wall surface of the measurement flow path 3a, and the exhaled air flow is changed.

Furthermore, another factor is considered to be that a part of the exhaled air flow colliding with the pressure resistance portion 40 enters the communication port 25 (first communication port 25A) communicating with the chamber 23 (first chamber 23A), and the measurement unit 10 (first measurement unit 10A) is caused to measure not only a static pressure but also a dynamic pressure.

As described above, the nasal respiration measurement device 1 according to the present embodiment includes the base member 3 in which the measurement flow path 3a that is in fluid communication with the nostril 111 of the user 100 is formed to pass through the base member 3, and the pressure resistance portion 40 that protrudes from the inner wall surface of the measurement flow path 3a. According to this configuration, the exhaled air flow in the nasal respiration of the user 100 is changed, and the respiratory flow can be measured with high accuracy,

In addition, in the nasal respiration measurement device 1 of the present embodiment, the pressure resistance portion 40 is formed integrally with the base member 3. According to this configuration, it is possible to reduce the cost by reducing the number of components.

In addition, in the nasal respiration measurement device 1 of the present embodiment, the pressure resistance portion 40 is provided in an annular shape over the entire inner wall surface of the measurement flow path 3a. According to this configuration, the exhaled air flow can be changed over the entire measurement flow path 3a.

In addition, in the nasal respiration measurement device 1 of the present embodiment, the communication port 25 communicating with the space in which the measurement unit 10 of the pressure sensor 2 is disposed is formed in the inner wall surface of the measurement flow path 3a. According to this configuration, the position where the pressure is measured can be easily changed depending on the position of the communication port 25 formed in the inner wall surface of the measurement flow path 3a.

In addition, in the nasal respiration measurement device 1 of the present embodiment, the pressure resistance portion 40 is disposed on the inner wall surface of the measurement flow path 3a on the downstream side of the communication port 25 (first communication port 25A) during exhalation. According to this configuration, since the signs of the pressure measured with the exhaled air flow and the pressure measured with the inhaled air flow is different, it is possible to determine not only the flow rate of respiration but also the exhaled air flow and the inhaled air flow.

It is noted that one or a plurality of the first communication ports 25A may be provided. In addition, one or a plurality of the second communication ports 25B may also be provided.

### Second Embodiment

Next, a second embodiment of the present invention is described. In the following description, the same or equivalent configurations as those in the above-described embodiment are denoted by the same reference numerals, and descriptions thereof are simplified or omitted.

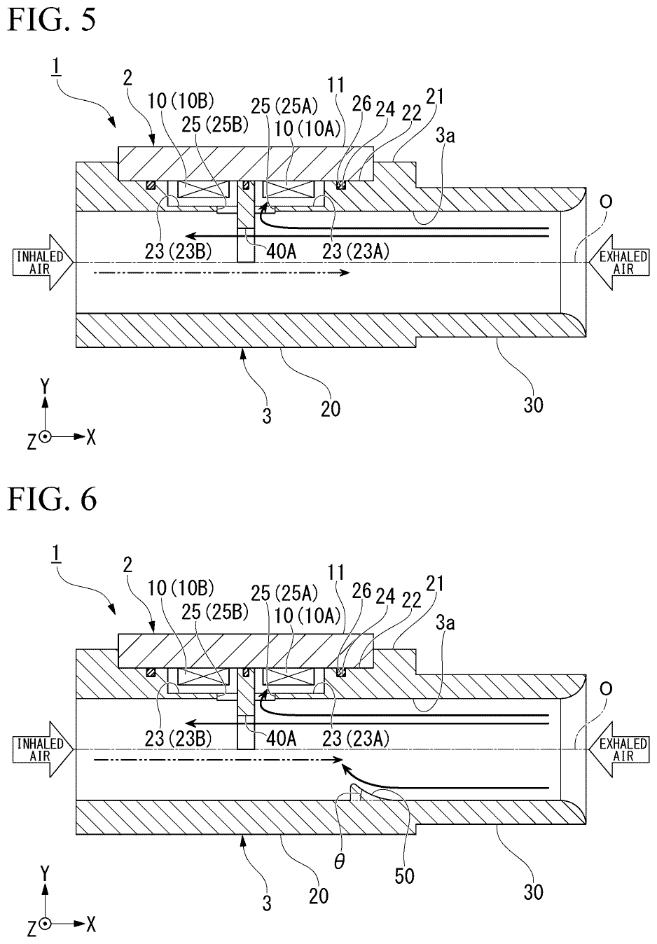

FIG. 5 is a cross-sectional configuration diagram of a nasal respiration measurement device 1 according to a second embodiment. It is noted that in FIG. 5, a cross-section of the same portion as that of FIG. 3 described above is shown. The same applies to FIGS. 6, 7, 9, and 10 described later.

As shown in FIG. 5, a pressure resistance portion 40A of the second embodiment is partially provided on the inner wall surface of the measurement flow path 3a on the side of the communication port 25. Specifically, the pressure resistance portion 40A is formed in an arc shape over the half of the inner wall surface on the +Y side of the inner wall surface of the measurement flow path 3a.

As described above, in the nasal respiration measurement device 1 of the second embodiment, the pressure resistance portion 40A is partially provided on the inner wall surface of the measurement flow path 3a on the side of the communication port 25.

According to this configuration, since the pressure resistance portion 40A is disposed only in the minimum necessary region on the inner wall surface of the measurement flow path 3a on the side of the communication port 25, the user 100 can easily breathe.

### Third Embodiment

Next, a third embodiment of the present invention is described. In the following description, the same or equivalent configurations as those in the above-described embodiment are denoted by the same reference numerals, and descriptions thereof are simplified or omitted.

FIG. 6 is a cross-sectional configuration diagram of a nasal respiration measurement device 1 according to a third embodiment.

As shown in FIG. 6, the nasal respiration measurement device 1 of the third embodiment includes an exhaled air flow guide portion 50 that guides exhaled air flowing on the inner wall surface of the measurement flow path 3a on the side (the −Y side) opposite to the communication port 25 to the side of communication port 25 (the +Y side).

The exhaled air flow guide portion 50 is provided on a side (−Y side) opposite to the communication port 25 and on an upstream side (+X side) of the communication port 25 (first communication port 25A) during exhalation. The exhaled air flow guide portion 50 has a slope shape in which the amount of protrusion in the radial direction with respect to the inner wall surface of the measurement flow path 3a gradually increases toward the-X side.

It is noted that the slope shape of the exhaled air flow guide portion 50 is preferably a curved surface shape so as not to disturb the exhaled air flow. That is, it is preferable that the exhaled air flow guide portion 50 has an inclined surface in which an inclination θ with respect to the inner wall surface of the measurement flow path 3a gradually increases from the +X side toward the −X side.

As described above, the nasal respiration measurement device 1 of the third embodiment includes the exhaled air flow guide portion 50 that is provided on the inner wall surface of the measurement flow path 3a on a side opposite to the communication port 25 and on the upstream side of the communication port 25 during exhalation and guides the exhaled air flowing on the side opposite to the communication port 25 to the side of the communication port 25. According to this configuration, on the inner wall surface of the measurement flow path 3a, the exhaled air flow biased toward the side opposite to the communication port 25 can be guided to the side of the communication port 25, so that measurement sensitivity of the exhaled air flow is improved.

### Fourth Embodiment

Next, a fourth embodiment of the present invention is described. In the following description, the same or equivalent configurations as those in the above-described embodiment are denoted by the same reference numerals, and descriptions thereof are simplified or omitted.

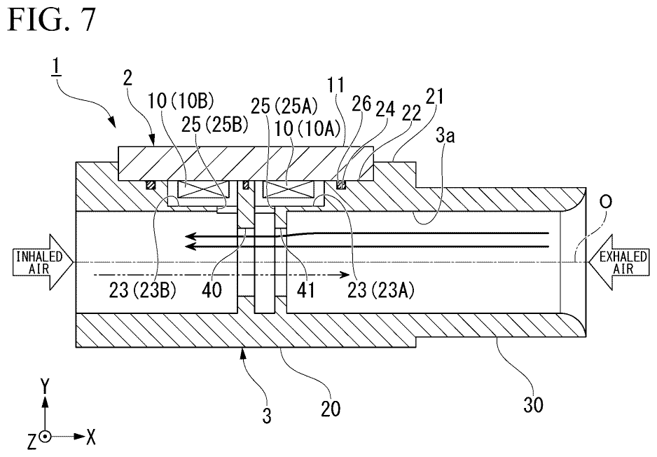

FIG. 7 is a cross-sectional configuration diagram of a nasal respiration measurement device 1 according to a fourth embodiment.

As shown in FIG. 7, the nasal respiration measurement device 1 of the fourth embodiment includes a plurality of pressure resistance portions 40 and 41 on the inner wall surface of the measurement flow path 3a.

The pressure resistance portion 40 is disposed on the downstream side of the first communication port 25A and on the upstream side of the second communication port 25B, as described above. The pressure resistance portion 41 is disposed on the inner wall surface of the measurement flow path 3a on the upstream side of the first communication port 25A during exhalation. That is, the plurality of pressure resistance portions 40 and 41 are provided on the inner wall surface of the measurement flow path 3a in series with respect to a fluid flow in the measurement flow path 3a.

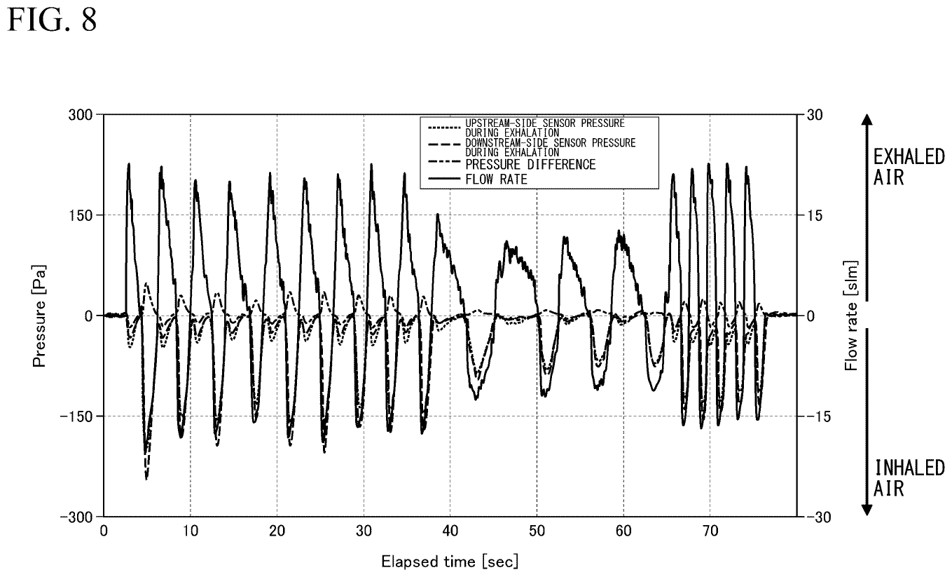

FIG. 8 is a view showing measurement results of the nasal respiration measurement device 1 according to the fourth embodiment. In FIG. 8, the left vertical axis indicates a pressure, the right vertical axis indicates the flow rate, and the horizontal axis indicates time. In a case where the pressure or the flow rate is positive (+), it is exhalation. In a case where the pressure or the flow rate is negative (−), it is inhalation.

In FIG. 8, “upstream-side sensor pressure during exhalation” means an output result of the first measurement unit 10A disposed on the upstream side of the measurement flow path 3a during exhalation. In addition, in FIG. 8, “downstream-side sensor pressure during exhalation” means an output result of the second measurement unit 10B disposed on the downstream side of the measurement flow path 3a during the exhalation. In addition, in FIG. 8, “pressure difference” means a difference between the output results of the first measurement unit 10A and the second measurement unit 10B. In addition, in FIG. 8, “flow rate” means the flow rate of the measurement flow path 3a which is measured by a flow meter as a reference.

In the nasal respiration measurement device 1 of the fourth embodiment, the plurality of pressure resistance portions 40 and 41 are provided on the inner wall surface of the measurement flow path 3a. According to this configuration, since the exhaled air flow can be changed at a plurality of places, measurement sensitivity of the exhaled air flow is improved. As a factor, it is considered that the pressure resistance portion 41 causes the exhaled air flow to be peeled from the inner wall surface of the measurement flow path 3a on the upstream side of the first communication port 25A, and thus the pressure of the exhaled air can be measured as the static pressure by the first measurement unit 10A.

In addition, the plurality of the pressure resistance portions 40 and 41 of the fourth embodiment are provided on the inner wall surface of the measurement flow path 3a in series with respect to a fluid flow of the measurement flow path 3a. According to this configuration, since the exhaled air flow can be changed at a plurality of places along the exhaled air flow, the measurement of the exhaled air flow is stabilized.

### Fifth Embodiment

Next, a fifth embodiment of the present invention is described. In the following description, the same or equivalent configurations as those in the above-described embodiment are denoted by the same reference numerals, and descriptions thereof are simplified or omitted.

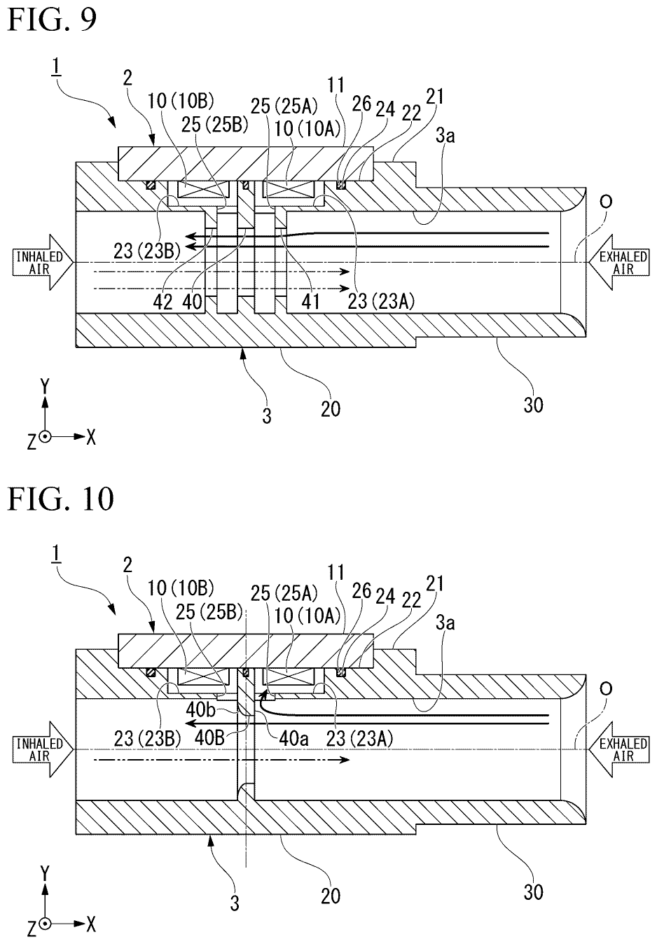

FIG. 9 is a cross-sectional configuration diagram of a nasal respiration measurement device 1 according to a fifth embodiment.

As shown in FIG. 9, the nasal respiration measurement device 1 of the fifth embodiment includes a plurality of pressure resistance portions 40, 41, and 42 on the inner wall surface of the measurement flow path 3a. The pressure resistance portion 42 is disposed on the inner wall surface of the measurement flow path 3a on a downstream side of the second communication port 25B during exhalation.

According to the fifth embodiment, since the pressure resistance portion 42 is provided on the downstream side of the second communication port 25B, the peeling action from the inner wall surface of the measurement flow path 3a is obtained by the pressure resistance portion 42 even in the inhaled air flow, and the pressure of the inhaled air flow can be measured as the static pressure by the second measurement unit 10B. Therefore, the measurement of the inhaled air flow is more stable.

In addition, the following effects can be obtained by changing inner diameters of the pressure resistance portions 40, 41, and 42. Specifically, in a case where the inner diameter of the pressure resistance portion 40 is set as D0, the inner diameter of the pressure resistance portion 41 is set as D1, and the inner diameter of the pressure resistance portion 42 is set as D2, the following (1) to (4) are satisfied.
(1) Case of D0=D1=D2

During exhalation, an output result of the first measurement unit 10A is a negative pressure, and an output result of the second measurement unit 10B is a negative pressure.

During inhalation, an output result of the first measurement unit 10A is a negative pressure, and an output result of the second measurement unit 10B is a negative pressure.
(2) Case of D0<D1=D2

In a case where the value of D1−D0 is positive during exhalation, an output result of the first measurement unit 10A is a positive pressure, and an output result of the second measurement unit 10B is a negative pressure.

During inhalation, an output result of the first measurement unit 10A is a negative pressure, and an output result of the second measurement unit 10B is a negative pressure.
(3) Case of D0>D1=D2

During exhalation, an output result of the first measurement unit 10A is a negative pressure, and an output result of the second measurement unit 10B is a negative pressure.

During inhalation, an output result of the first measurement unit 10A is a negative pressure, and an output result of the second measurement unit 10B is a negative pressure.
(4) Case of D2>D0>D1

During exhalation, an output result of the first measurement unit 10A is a negative pressure, and an output result of the second measurement unit 10B is a negative pressure.

During inhalation, an output result of the first measurement unit 10A is a negative pressure, and an output result of the second measurement unit 10B is a negative pressure.
(5) Case of D2<D0<D1

During exhalation, an output result of the first measurement unit 10A is a negative pressure, and an output result of the second measurement unit 10B is a negative pressure.

Alternatively, in a case where the value of D1−D0 is positive, an output result of the first measurement unit 10A is a positive pressure, and in a case where the value of D0−D2 is positive, an output result of the second measurement unit 10B is a positive pressure.

During inhalation, an output result of the first measurement unit 10A is a negative pressure, and an output result of the second measurement unit 10B is a negative pressure.

### Sixth Embodiment

Next, a sixth embodiment of the present invention is described. In the following description, the same or equivalent configurations as those in the above-described embodiment are denoted by the same reference numerals, and descriptions thereof are simplified or omitted.

FIG. 10 is a cross-sectional configuration diagram of a nasal respiration measurement device 1 according to a sixth embodiment.

As shown in FIG. 10, in the nasal respiration measurement device 1 of the sixth embodiment, the pressure resistance portion 40B has an asymmetric shape between the upstream side and the downstream side thereof.

Specifically, a corner portion of the pressure resistance portion 40B on the upstream side (+X side) during exhalation is a right angle, and a surface of the pressure resistance portion 40B facing the upstream side (+X side) during exhalation is the flat surface 40a. In addition, a corner portion of the pressure resistance portion 40B on the downstream side (−X side) during exhalation is rounded, and a surface of the pressure resistance portion 40B facing the downstream side (−X side) during exhalation is a curved surface 40b.

As described above, in the nasal respiration measurement device 1 of the sixth embodiment, the pressure resistance portion 40 has an asymmetric shape between the upstream side and the downstream side thereof. According to this configuration, it is possible to apply different rectification (change in flow) actions to the exhaled air flow and the inhaled air flow. For example, an effect of introducing the exhaled air flow into the first communication port 25A to measure the dynamic pressure is obtained on the upstream side (+X side) of the pressure resistance portion 40B during exhalation. In addition, on the upstream side (−X side) of the pressure resistance portion 40B during inhalation, an action of reducing the resistance of the inhaled air flow and facilitating respiration is obtained.

As described above, the nasal respiration measurement device 1 of the sixth embodiment includes the base member 3 in which the measurement flow path 3a that is in fluid communication with the nostril 111 of the user 100 is formed to pass through the base member 3, and the pressure resistance portion 40 that protrudes from the inner wall surface of the measurement flow path 3a, in which the communication port 25 communicating with the space in which the pressure sensor 2 is disposed is formed in the inner wall surface of the measurement flow path 3a, and the pressure resistance portion 40 is disposed on the inner wall surface of the measurement flow path 3a on the downstream side of the communication port 25 during exhalation, and the pressure resistance portion 40 has an asymmetric shape between the upstream side and the downstream side thereof. According to this configuration, since the exhalation and inhalation in the nasal respiration of the user 100 can be changed differently, the respiratory flow can be measured with high accuracy.

In addition, the configuration of the sixth embodiment may be combined with the configuration of the third embodiment. In this case, the nasal respiration measurement device 1 includes the base member 3 in which the measurement flow path 3a that is in fluid communication with a nostril 111 of the user 100 is formed to pass through the base member 3, a pressure resistance portion 40 that protrudes from an inner wall surface of the measurement flow path 3a, and a communication port 25 that is formed in the inner wall surface of the measurement flow path 3a and communicates with a space in which the pressure sensor 2 is disposed. The pressure resistance portion 40 is disposed on the inner wall surface of the measurement flow path 3a on a downstream side of the communication port 25 during exhalation and is partially provided on the inner wall surface of the measurement flow path 3a on the side of the communication port 25. The pressure resistance portion 40 has an asymmetric shape on the upstream side and the downstream side thereof.

According to this configuration, the exhaled air flow in the nasal respiration of the user 100 is changed, and the respiratory flow can be measured with high accuracy.

### Seventh Embodiment

Next, a seventh embodiment of the present invention is described. In the following description, the same or equivalent configurations as those in the above-described embodiment are denoted by the same reference numerals, and descriptions thereof are simplified or omitted.

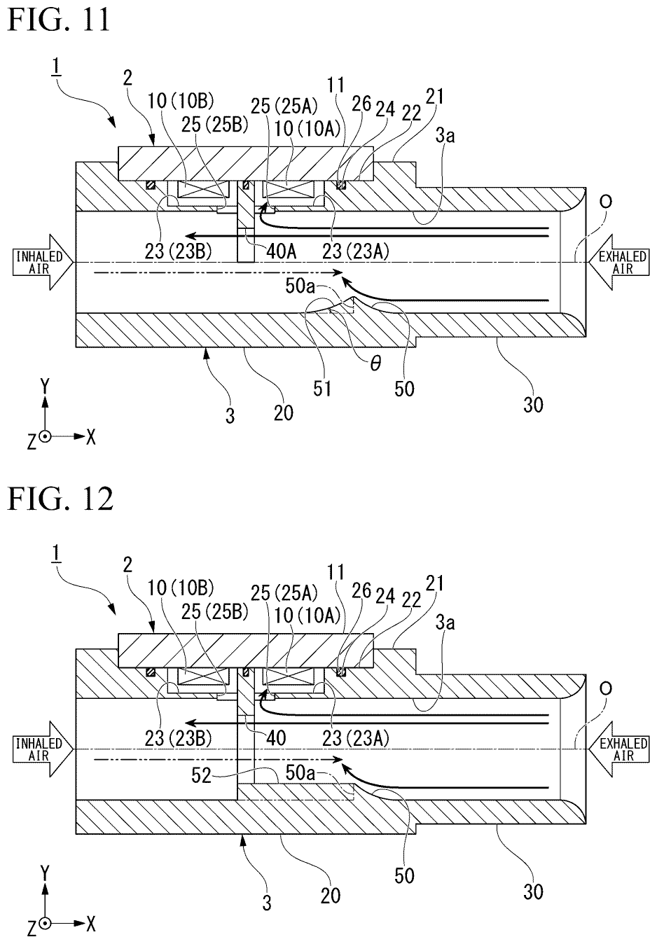

FIG. 11 is a cross-sectional configuration diagram of a nasal respiration measurement device 1 according to the seventh embodiment.

As shown in FIG. 11, in the nasal respiration measurement device 1 of the seventh embodiment, a slope 51 is provided on a side of a rear surface 50a (inhalation side) of the exhaled air flow guide portion 50.

Specifically, the slope 51 is disposed on the-X side of the exhaled air flow guide portion 50 and is installed consecutively to the exhaled air flow guide portion 50. The slope 51 has an inclined surface in which an inclination θ with respect to the inner wall surface of the measurement flow path 3a gradually increases from the −X side toward the +X side.

According to this configuration, since the inhaled air flow smoothly climbs over the exhaled air flow guide portion 50 by the slope 51 without colliding with the rear surface 50a. (indicated by a one-dot chain line in FIG. 11) of the exhaled air flow guide portion 50, it is possible to mitigate the pressure resistance during inhalation in the measurement flow path 3a. That is, even in a case where the exhaled air flow guide portion 50 is provided, the user can easily breathe.

It is noted that the slope 51 shown in FIG. 11 has a shape that is opposite to the exhaled air flow guide portion 50 and is similar to the exhaled air flow guide portion 50, but may have a shape different from the exhaled air flow guide portion 50. For example, at least one of the slope 51 or the exhaled air flow guide portion 50 may have an inclined shape in which the inclination θ is constant.

### Eighth Embodiment

Next, an eighth embodiment of the present invention is described. In the following description, the same or equivalent configurations as those in the above-described embodiment are denoted by the same reference numerals, and descriptions thereof are simplified or omitted.

FIG. 12 is a cross-sectional configuration diagram of a nasal respiration measurement device 1 according to the eighth embodiment.

As shown in FIG. 12, in the nasal respiration measurement device 1 of the eighth embodiment, the exhaled air flow guide portion 50 is installed consecutively to the pressure resistance portion 40.

Specifically, a consecutive installation portion 52 extending in the axial direction (X-axis direction) from the side of the rear surface 50a of the exhaled air flow guide portion 50 to the pressure resistance portion 40 is formed on the inner wall surface of the measurement flow path 3a. That is, the exhaled air flow guide portion 50 and the pressure resistance portion 40 are formed integrally. According to this configuration, a recess portion (indicated by a one-dot chain line in FIG. 12) may not be formed between the pressure resistance portion 40 and the exhaled air flow guide portion 50, so that it is easy to manufacture the base member 3 (for example, by injection molding of the base member 3), and the manufacturing cost can be reduced.

### Ninth Embodiment

Next, a ninth embodiment of the present invention is described. In the following description, the same or equivalent configurations as those in the above-described embodiment are denoted by the same reference numerals, and descriptions thereof are simplified or omitted.

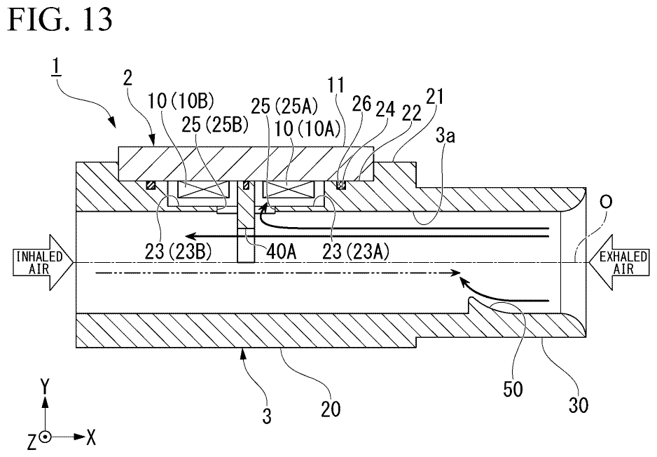

FIG. 13 is a cross-sectional configuration diagram of a nasal respiration measurement device 1 according to the ninth embodiment.

As shown in FIG. 13, in the nasal respiration measurement device 1 of the ninth embodiment, the exhaled air flow guide portion 50 is disposed inside the insertion portion 30.

Specifically, the base member 3 includes the insertion portion 30 that is to be inserted into the nostril 111 (see FIG. 1). The exhaled air flow guide portion 50 is disposed inside the insertion portion 30. That is, the exhaled air flow guide portion 50 is disposed in the vicinity of an upstream end portion of the measurement flow path 3a such that the exhaled air flow is likely to collide with the exhaled air flow guide portion 50. According to this configuration, since the exhaled air flow guide portion 50 guides the exhaled air flow flowing into the measurement flow path 3a to the side of the communication port 25 immediately, the measurement sensitivity of the exhaled air flow is improved.

### Tenth Embodiment

Next, a tenth embodiment of the present invention is described. In the following description, the same or equivalent configurations as those in the above-described embodiment are denoted by the same reference numerals, and descriptions thereof are simplified or omitted.

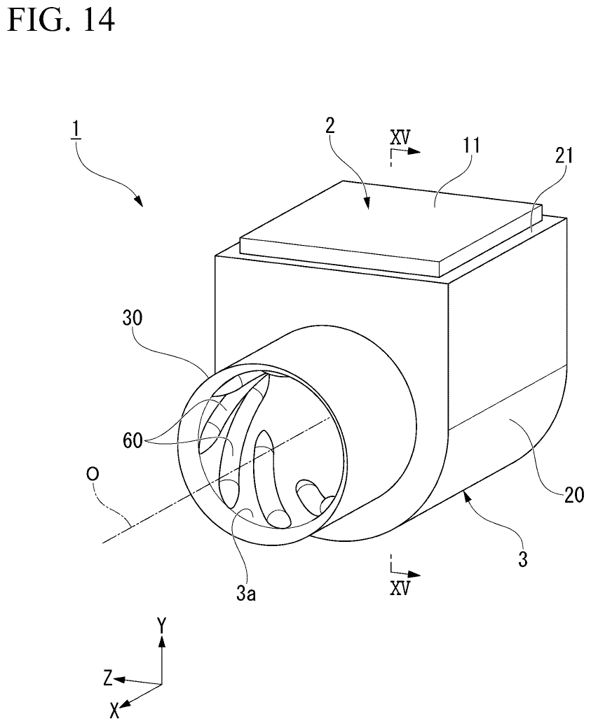

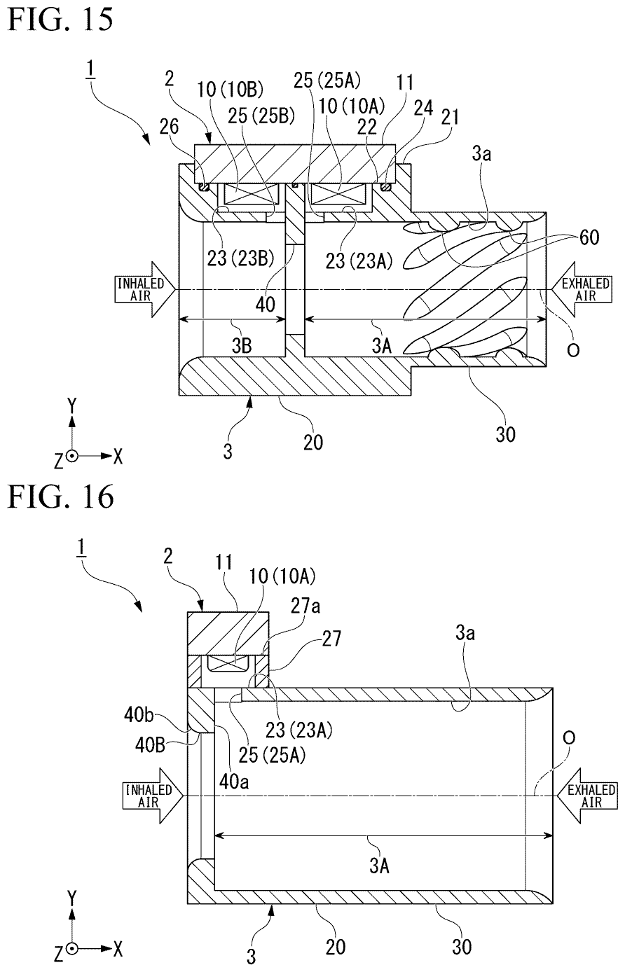

FIG. 14 is a perspective view of a nasal respiration measurement device 1 according to the tenth embodiment. FIG. 15 is a cross-sectional configuration diagram of the nasal respiration measurement device 1 according to the tenth embodiment.

As shown in FIGS. 14 and 15, in the nasal respiration measurement device 1 of the tenth embodiment, a spiral protrusion 60 is formed on the inner wall surface of the measurement flow path 3a.

As shown in FIG. 15, the measurement flow path 3a includes an upstream region 3A formed on the upstream side of the pressure resistance portion 40 during exhalation and a downstream region 3B formed on the downstream side of the pressure resistance portion 40 during exhalation. The flow path length of the downstream region 3B is shorter than the flow path length of the upstream region 3A. Since the downstream region 3B is less likely to affect measurement of the exhaled air flow, the nasal respiration measurement device 1 can be reduced in size by making the flow path length of the downstream region 3B shorter than the flow path length of the upstream region 3A. It is noted that the nasal respiration measurement device 1 of each of the above-described embodiments also has the same configuration.

A spiral protrusion 60 is formed on the inner wall surface of the upstream region 3A. The spiral protrusion 60 is disposed inside the insertion portion 30. That is, the spiral protrusion 60 is disposed in the vicinity of the upstream end portion of the measurement flow path 3a such that the exhaled air flow is likely to collide. According to this configuration, since a biased exhaled air flow along the inner wall surface of the measurement flow path 3a can be stirred by the spiral protrusion 60, the measurement sensitivity of the exhaled air flow is improved. In addition, according to this configuration, it is possible to apply different rectifying (change in flow) actions to the upstream region 3A and the downstream region 3B based on the pressure resistance portion 40.

### Eleventh Embodiment

Next, an eleventh embodiment of the present invention is described. In the following description, the same or equivalent configurations as those in the above-described embodiment are denoted by the same reference numerals, and descriptions thereof are simplified or omitted.

FIG. 16 is a cross-sectional configuration diagram of a nasal respiration measurement device 1 according to the eleventh embodiment.

As shown in FIG. 16, the nasal respiration measurement device 1 of the eleventh embodiment includes a substrate portion 11 disposed in parallel with the axial direction in which the central axis O of the measurement flow path 3a extends.

Specifically, the substrate portion 11 is formed in a flat plate shape in which a thickness direction matches the Y-axis direction and extends along an X-Z plane. The substrate portion 11 is attached to an upper surface 27a of a substrate support portion 27 provided on the base member 3. The chamber 23 is formed inside the substrate support portion 27. According to this configuration, since the axial direction (X-axis direction) of the measurement flow path 3a and the plane direction of the substrate portion 11 match each other, a dimension in the radial direction (for example, the Y-axis direction) orthogonal to the central axis O of the nasal respiration measurement device 1 can be reduced.

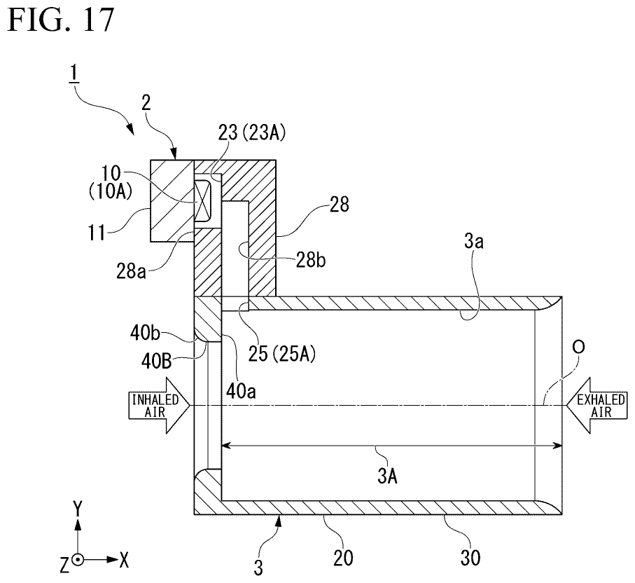

In addition, in the nasal respiration measurement device 1 shown in FIG. 16 and FIG. 17 to be described later, there is no downstream region 3B with respect to the pressure resistance portion 40B. Accordingly, the dimension of the nasal respiration measurement device 1 in the X-axis direction can be reduced.

### Twelfth Embodiment

Next, a twelfth embodiment of the present invention is described, In the following description, the same or equivalent configurations as those in the above-described embodiment are denoted by the same reference numerals, and descriptions thereof are simplified or omitted.

FIG. 17 is a cross-sectional configuration diagram of a nasal respiration measurement device 1 according to the twelfth embodiment.

As shown in FIG. 17, the nasal respiration measurement device 1 of the twelfth embodiment includes a substrate portion 11 that is disposed perpendicular to the axial direction in which the central axis O of the measurement flow path 3a extends.

Specifically, the substrate portion 11 is formed in a flat plate shape in which a thickness direction matches the X-axis direction and extends along the Y-Z plane. The substrate portion 11 is attached to a side surface 28a of a substrate support portion 28 provided on the base member 3, the side surface 28a facing the −X side. The chamber 23 and a connection flow path 28b that connects the chamber 23 and the communication port 25 are formed inside the substrate support portion 28. According to this configuration, since the axial direction (X-axis direction) of the measurement flow path 3a and the thickness direction of the substrate portion 11 match each other, the dimension of the nasal respiration measurement device 1 in the axial direction can be reduced.

### Thirteenth Embodiment

Next, a thirteenth embodiment of the present invention is described. In the following description, the same or equivalent configurations as those in the above-described embodiment are denoted by the same reference numerals, and descriptions thereof are simplified or omitted.

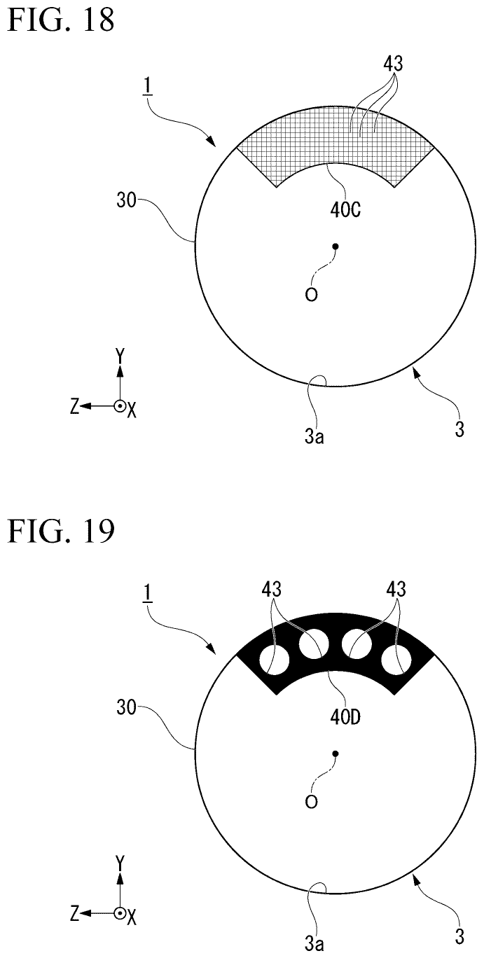

FIG. 18 is a cross-sectional configuration diagram of a nasal respiration measurement device 1 according to the thirteenth embodiment.

As shown in FIG. 18, the nasal respiration measurement device 1 of the thirteenth embodiment includes a pressure resistance portion 40C (mesh type) in which one or a plurality of through-holes 43 are formed.

Specifically, the pressure resistance portion 40C is formed in a mesh shape and includes a plurality of through-holes 43 that penetrate in the X-axis direction. According to this configuration, even in a case where the pressure resistance portion 40C is provided, since the through-holes 43 are formed in the pressure resistance portion 40, the user can easily breathe.

FIG. 19 is a cross-sectional configuration diagram of a nasal respiration measurement device 1 according to a modification example of the thirteenth embodiment.

A pressure resistance portion 40D (through-hole type) shown in FIG. 19 is formed in an arc plate shape and includes a plurality of through-holes 43 that penetrate in the X-axis direction. Even with this configuration, since the through-holes 43 are formed in the pressure resistance portion 40D, the user can easily breathe. It is noted that only one through-hole 43 may be provided. In addition, the through-holes 43 are not limited to a circular shape and may have a polygonal shape, a circular arc shape, an elliptical shape, or other shapes.

### Fourteenth Embodiment

Next, a fourteenth embodiment of the present invention is described. In the following description, the same or equivalent configurations as those in the above-described embodiment are denoted by the same reference numerals, and descriptions thereof are simplified or omitted.

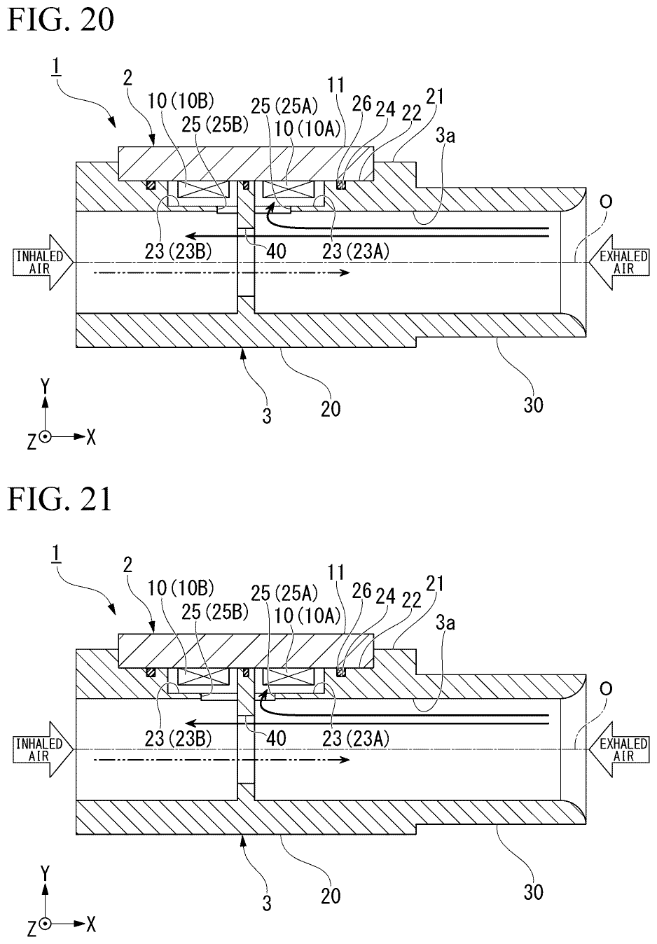

FIG. 20 is a cross-sectional configuration diagram of a nasal respiration measurement device 1 according to the fourteenth embodiment.

As shown in FIG. 20, in the nasal respiration measurement device 1 of the fourteenth embodiment, a pressure loss of the first communication port 25A and a pressure loss of the second communication port 25B are different from each other.

Specifically, an opening area of the first communication port 25A is larger than an opening area of the second communication port 25B. Since the first communication port 25A has a positive pressure during exhalation, in a case where the pressure loss of the first communication port 25A is lower than the pressure loss of the second communication port 25B, the exhaled air is more likely to enter the first communication port 25A, and the sensitivity during exhalation is increased.

FIG. 21 is a cross-sectional configuration diagram of a nasal respiration measurement device 1 according to one modification example of the fourteenth embodiment.

The opening area of the first communication port 25A shown in FIG. 21 is smaller than the opening area of the second communication port 25B. In a case where the pressure resistance of the pressure resistance portion 40 is unintentionally increased due to an influence of nasal mucus or the like, the pressure applied to the first measurement unit 10A during exhalation is increased, and an unintentional large pressure may be applied to the pressure sensor 2. In a case where the pressure loss of the first communication port 25A is greater than the pressure loss of the second communication port 25B, the exhaled air is less likely to enter the first communication port 25A, and a damage to the pressure sensor 2 can be prevented. It is noted that in addition to the opening area, a length (flow path length) of the opening or a friction coefficient of the inner wall surface of the opening may be changed as a method of making the pressure loss of the first communication port 25A and the pressure loss of the second communication port 25B different from each other.

While preferred embodiments of the present invention are described and illustrated above, it should be understood that these are exemplary examples of the present invention and should not be considered as limiting. Additions, omissions, substitutions, and other changes can be made without departing from the scope of the present invention. Accordingly, the present invention should not be considered as being limited by the foregoing description, and is only limited by the scope of the appended claims.

For example, in the above-described embodiment, the pressure resistance portion is provided in series with the exhaled air flow (inhaled air flow), but the rectifying action can be obtained even in a case where the pressure resistance portion is provided in parallel with the exhaled air flow (inhaled air flow).

In addition, for example, the combination and substitution of the components of the above-described embodiments can be appropriately changed.

What is claimed is:

1. A nasal respiration measurement device, comprising:
a base member in which a measurement flow path that is in fluid communication with a nostril of a user is formed to pass through the base member; and
a pressure resistance portion that protrudes from an inner wall surface of the measurement flow path.

2. The nasal respiration measurement device according to claim 1,
wherein the pressure resistance portion is formed integrally with the base member.

3. The nasal respiration measurement device according to claim 1,
wherein a plurality of the pressure resistance portions are provided on the inner wall surface of the measurement flow path.

4. The nasal respiration measurement device according to claim 1,
wherein a plurality of the pressure resistance portions are provided on the inner wall surface of the measurement flow path in series with respect to a fluid flow of the measurement flow path.

5. The nasal respiration measurement device according to claim 1,
wherein the pressure resistance portion is provided in an annular shape over an entire inner wall surface of the measurement flow path.

6. The nasal respiration measurement device according to claim 1,
wherein the pressure resistance portion has an asymmetric shape between an upstream side and a downstream side thereof.

7. The nasal respiration measurement device according to claim 1,
wherein a communication port communicating with a space in which a pressure sensor is disposed is formed in the inner wall surface of the measurement flow path.

8. The nasal respiration measurement device according to claim 7,
wherein the pressure sensor includes a substrate portion disposed perpendicular to an axial direction in which a central axis of the measurement flow path extends.

9. The nasal respiration measurement device according to claim 7,
wherein the pressure sensor includes a substrate portion disposed in parallel with an axial direction in which a central axis of the measurement flow path extends.

10. The nasal respiration measurement device according to claim 7,
wherein the measurement flow path includes, as the communication port, one or a plurality of first communication ports that are disposed in an upstream region on an upstream side of the pressure resistance portion during exhalation and communicate with a space in which a first measurement unit of the pressure sensor is disposed.

11. The nasal respiration measurement device according to claim 10,
wherein the measurement flow path includes, as the communication port, one or a plurality of second communication ports that are disposed in a downstream region on a downstream side of the pressure resistance portion during exhalation and communicate with a space in which a second measurement unit of the pressure sensor is disposed.

12. The nasal respiration measurement device according to claim 11,
wherein a pressure loss of the first communication port and a pressure loss of the second communication port are different from each other.

13. The nasal respiration measurement device according to claim 7,
wherein the pressure resistance portion is partially provided on the inner wall surface of the measurement flow path on a side of the communication port.

14. The nasal respiration measurement device according to claim 7, further comprising:
an exhaled air flow guide portion that is provided on the inner wall surface of the measurement flow path on a side opposite to the communication port and on an upstream side of the communication port during exhalation, and configured to guide exhaled air flowing on the side opposite to the communication port to a side of the communication port.

15. The nasal respiration measurement device according to claim 14,
wherein a slope that mitigates pressure resistance of the exhaled air flow guide portion during inhalation is provided on the inner wall surface of the measurement flow path.

16. The nasal respiration measurement device according to claim 14,
wherein the exhaled air flow guide portion is installed consecutively to the pressure resistance portion.

17. (canceled)

18. The nasal respiration measurement device according to claim 1,
wherein the measurement flow path includes an upstream region on an upstream side of the pressure resistance portion during exhalation and a downstream region on a downstream side of the pressure resistance portion during exhalation, and
a flow path length of the downstream region is shorter than a flow path length of the upstream region.

19. (canceled)

20. The nasal respiration measurement device according to claim 1,
wherein one or a plurality of through-holes are formed in the pressure resistance portion.

21. A nasal respiration measurement device, comprising:
a base member in which a measurement flow path that is in fluid communication with a nostril of a user is formed to pass through the base member; and
a pressure resistance portion that protrudes from an inner wall surface of the measurement flow path,
wherein a communication port communicating with a space in which a pressure sensor is disposed is formed in the inner wall surface of the measurement flow path, and
the pressure resistance portion has an asymmetric shape between an upstream side and a downstream side thereof.

22. A nasal respiration measurement device, comprising:
a base member in which a measurement flow path that is in fluid communication with a nostril of a user is formed to pass through the base member; and
a pressure resistance portion that protrudes from an inner wall surface of the measurement flow path,
wherein a communication port communicating with a space in which a pressure sensor is disposed is formed in the inner wall surface of the measurement flow path,
the pressure resistance portion is disposed on the inner wall surface of the measurement flow path on a downstream side of the communication port during exhalation, and
the pressure resistance portion has an asymmetric shape between an upstream side and a downstream side thereof.

23. (canceled)
